(19) United States
(12) Patent Application Publication
(10) Pub. No.: US 2024/0374928 A1
(43) Pub. Date: Nov. 14, 2024

(54) PRE-TRAINING METHOD, PRE-TRAINING SYSTEM, TRAINING METHOD, AND TRAINING SYSTEM FOR DOSE DISTRIBUTION PREDICTION MODEL

(71) Applicant: SHANGHAI UNITED IMAGING HEALTHCARE CO., LTD., Shanghai (CN)

(72) Inventor: Kejun ZHAO, Shanghai (CN)

(73) Assignee: SHANGHAI UNITED IMAGING HEALTHCARE CO., LTD., Shanghai (CN)

(21) Appl. No.: 18/661,588

(22) Filed: May 11, 2024

(30) Foreign Application Priority Data
May 11, 2023 (CN) 202310533773.5

(51) Int. Cl. A61N 5/10; G16H 20/40

(52) U.S. Cl. CPC A61N 5/1031; G16H 20/40

(57) ABSTRACT

The present disclosure provides a pre-training method for a dose distribution prediction model, implemented on a device including at least one processor and at least one storage device. The method comprises: obtaining a plurality of training tasks; for each of the plurality of training tasks, obtaining one or more samples corresponding to the training task; and obtaining a pre-training model based on the samples corresponding to the plurality of training tasks. The pre-training model is configured to obtain a dose distribution prediction model for a target task by adjusting, based on one or more samples corresponding to the target task, the pre-training model. The dose distribution prediction model is configured to output predicted dose distribution information corresponding to the target task. For each of the samples of the plurality of tasks and the target task, a label of the sample includes labeled dose distribution information corresponding to the task.

### CROSS-REFERENCE TO RELATED APPLICATIONS

This application claims priority of Chinese Application No. 202310533773.5 filed on May 11, 2023, the contents of which are incorporated herein by reference to its entirety.

### TECHNICAL FIELD

The present disclosure relates to the field of machine learning, and in particular to a pre-training method, a pre-training system, a training method, and a training system for a dose distribution prediction model.

### BACKGROUND

Radiation therapy (also referred to as radiotherapy) is one of the most important means of cancer treatment today, and the main objective of radiation therapy is to ensure that a target region receives a prescribed dose of radiation and to reduce a dose of radiation received by surrounding normal organs. With the development of advanced treatments such as Intensity-Modulated Radiation Therapy (IMRT) and Volumetric-Modulated Arc Therapy (VMAT), the quality of radiotherapy plans has been significantly improved. However, the development of a high-quality radiotherapy plan usually requires significant manual intervention, whereby physicists need to repeatedly adjust the plan parameters based on their own experience and feedback from physicians, resulting in an elongated time of plan development, which is detrimental to tumor control and patient survival. With the development of machine learning technology, the dose distribution in a patient's body can be predicted (referred to as dose prediction) using a machine learning model (e.g., a deep learning model). Dose prediction can provide a reasonable initial optimization target, which can effectively shorten the development time of radiotherapy plans. However, some current machine learning programs for achieving dose prediction are poorly generalized and difficult to adapt to different targets (or referred to as tasks). For example, applying a dose distribution prediction model originally used for a nasopharyngeal site to dose prediction at a prostate site tends to be poor.

### SUMMARY

The embodiments of the present disclosure provide a pre-training method for a dose distribution prediction model, implemented on a device including at least one processor and at least one storage device. The method may comprise: obtaining a plurality of training tasks; for each of the plurality of training tasks, obtaining one or more samples corresponding to the training task; and obtaining a pre-training model based on the samples corresponding to the plurality of training tasks. The pre-training model may be configured to obtain a dose distribution prediction model for a target task by adjusting, based on one or more samples corresponding to the target task, the pre-training model. The dose distribution prediction model may be configured to output predicted dose distribution information corresponding to the target task. For each of the samples of the plurality of tasks and the target task, a label of the sample may include labeled dose distribution information corresponding to the task.

The embodiments of the present disclosure provide a pre-training system for a dose distribution prediction model. The pre-training system may comprise at least one storage device including a set of instructions; and at least one processor in communication with the at least one storage device. When executing the set of instructions, the at least one processor may be directed to perform operations including: obtaining a plurality of training tasks; for each of the plurality of training tasks, obtaining one or more samples corresponding to the training task; and obtaining a pre-training model based on the samples corresponding to the plurality of training tasks, the pre-training model being configured to obtain a dose distribution prediction model for a target task by adjusting, based on one or more samples corresponding to the target task, the pre-training model, the dose distribution prediction model being configured to output predicted dose distribution information corresponding to the target task. For each of the samples of the plurality of tasks and the target task, a label of the sample may include labeled dose distribution information corresponding to the task.

The embodiments of the present disclosure provide a training method for a dose distribution prediction model, implemented on a device including at least one processor and at least one storage device. The method may comprise: obtaining a pre-training model; obtaining one or more samples corresponding to a target task, a label of each of the one or more samples corresponding to the target task including labeled dose distribution information corresponding to the task; and obtaining a dose distribution prediction model for the target task by adjusting, based on the one or more samples, the pre-training model, the dose distribution prediction model for the target task being configured to output predicted dose distribution information corresponding to the target task.

### BRIEF DESCRIPTION OF THE DRAWINGS

The present disclosure will be further illustrated by way of exemplary embodiments, which will be described in detail with reference to the accompanying drawings. These embodiments are not limiting, and in these embodiments, the same numbering denotes the same structure, wherein:

FIG. 1 is a schematic diagram illustrating an application scenario of an exemplary treatment plan system according to some embodiments of the present disclosure;

FIG. 2 is a flowchart illustrating an exemplary pre-training method for a dose distribution prediction model according to some embodiments of the present disclosure;

FIG. 3 is a flowchart illustrating an exemplary pre-training phase according to some embodiments of the present disclosure;

FIG. 4 is a flowchart illustrating an exemplary training method for a dose distribution prediction model according to some embodiments of the present disclosure;

FIG. 5 is a schematic diagram illustrating a data flow of an exemplary conditional generative adversarial network according to some embodiments of the present disclosure

FIG. 6 is a schematic diagram illustrating a structure of an exemplary conditional adversarial network according to some embodiments of the present disclosure;

FIG. 7 is a schematic diagram illustrating a structure of an exemplary generator according to some embodiments of the present disclosure;

FIG. 8 is a block diagram illustrating an exemplary pre-training system for a dose distribution prediction model according to some embodiments of the present disclosure; and

FIG. 9 is a block diagram illustrating an exemplary training system for a dose distribution prediction model according to some embodiments of the present disclosure.

### DETAILED DESCRIPTION

In order to more clearly illustrate the technical solutions of the embodiments of the present disclosure, the following briefly introduces the drawings that need to be used in the description of the embodiments. Apparently, the accompanying drawings in the following description are only some examples or embodiments of the present disclosure, and those skilled in the art can also apply the present disclosure to other similar scenarios according to the drawings without creative efforts. Unless obviously obtained from the context or the context illustrates otherwise, the same numeral in the drawings refers to the same structure or operation.

It should be understood that “system”, “device”, “unit” and/or “module” as used herein is a method for distinguishing different components, elements, parts, portions or assemblies of different levels. However, the words may be replaced by other expressions if other words can achieve the same purpose.

As indicated in the disclosure and claims, the terms “a”, “an” and/or “the” are not specific to the singular form and may include the plural form unless the context clearly indicates an exception. Generally speaking, the terms “comprising” and “including” only suggest the inclusion of clearly identified steps and elements, and these steps and elements do not constitute an exclusive list, and the method or device may also contain other steps or elements.

The technical problem to be solved by the present disclosure is to provide a dose prediction method applicable to multiple targets (tasks). The present disclosure is desirable to provide a training method to generate a dose distribution prediction model that adapts to different tasks. The flowchart is used in the present disclosure to illustrate the operations performed by the system according to the embodiments of the present disclosure. It should be understood that the preceding or following operations are not necessarily performed in the exact order. Instead, various steps may be processed in reverse order or simultaneously. Meanwhile, other operations may be added to these procedures, or a certain step or steps may be removed from these procedures.

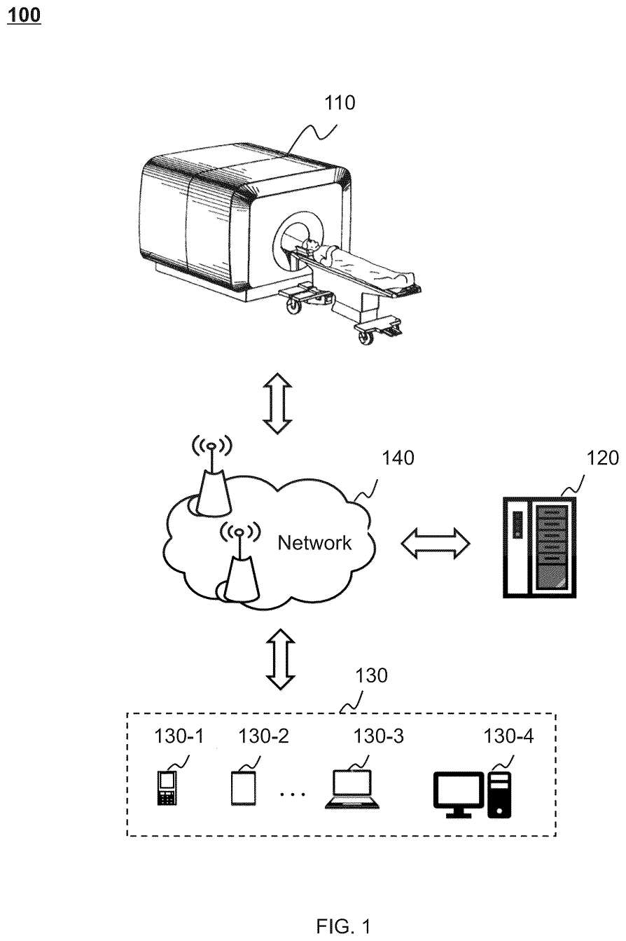

FIG. 1 is a schematic diagram illustrating an application scenario of an exemplary treatment plan system according to some embodiments of the present disclosure. As illustrated in FIG. 1, a system 100 may include a data acquisition device 110, a processing device 120, a user terminal 130, and a network 140.

The data acquisition device 110 may be configured to acquire relevant data of a subject (for example, a patient or an animal). The relevant data can refer to data related to dose prediction. In some embodiments, the relevant data of the subject may include medical imaging data of the subject, such as a full-body scan image of the subject, an image of a region of interest (ROI) of the subject, etc.

In some embodiments, the data acquisition device 110 may include an imaging device. The imaging device may include a single modality imaging device and/or a multi-modality imaging device. The single modality imaging device may include, for example, a computed tomography (CT) device, a positron emission computed tomography (PET) device, a magnetic resonance imaging (MRI) device, an ultrasonic imaging device, an X-ray imaging device, a single photon emission computed tomography (SPECT) device, etc.

The multi-modality imaging device may include, for example, an MRI-CT device, a PET-MRI device, SPECT-MRI device, a digital subtraction angiography-magnetic resonance imaging (DSA-MRI) device, a PET-CT device, an SPECT-CT device, etc.

In some embodiments, the system 100 may include a radiotherapy device. The radiotherapy device may include a treatment plan system (TPS), image-guide radiotherapy (IGRT), etc. The image-guide radiotherapy (IGRT) may include a treatment device and an imaging device. The treatment device may include a linear accelerator, a cyclotron, a synchrotron, etc., configured to perform a radio therapy on a subject. The treatment device may include an accelerator of species of particles including, for example, photons, electrons, protons, or heavy ions. The imaging device may include a device similar to the data acquisition device 110, or may include an electronic portal imaging device (EPID), etc.

The processing device 120 may be configured to generate a treatment plan, such as a radiotherapy plan. Specifically, the processing device 120 may obtain the relevant data of the subject from the data acquisition device 110, input the relevant data of the subject into a dose distribution prediction model obtained through training to obtain predicted dose distribution information of the subject. The processing device 120 may generate the treatment plan for the subject based on the predicted dose distribution information of the subject.

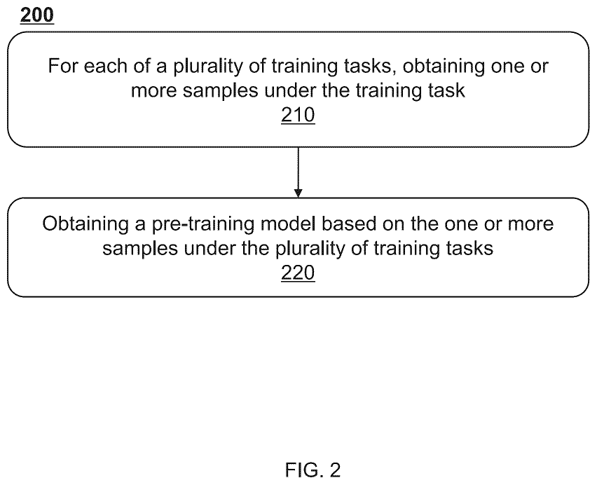

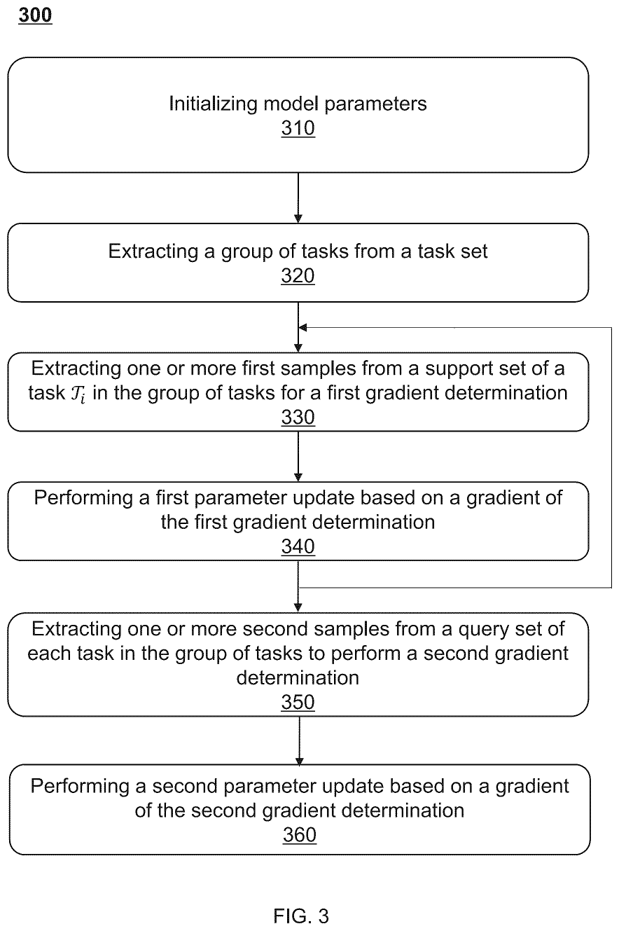

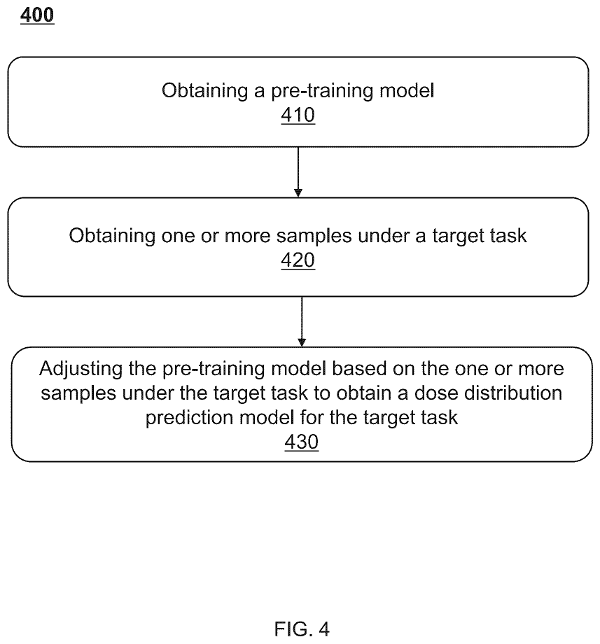

The processing device 120 may also be configured to obtain the dose distribution prediction model. A process of obtaining the dose distribution prediction model may be divided into a pre-training phase and a fine-tuning phase. More descriptions regarding the pre-training phase may be found in FIGS. 2-3 and related descriptions thereof. More descriptions regarding the fine-tuning phase may be found in FIG. 4 and related descriptions thereof.

In some embodiments, the processing device 120 may be a single server or a group of servers. The group of servers may be centralized or distributed. In some embodiments, the processing device 120 may be local or remote. In some embodiments, the processing device 120 may be implemented on a cloud platform. Merely by way of example, the cloud platform may include a private cloud, a public cloud, a hybrid cloud, a community cloud, a distributed cloud, an internal cloud, a multilayer cloud, or the like, or any combination thereof.

The user terminal 130 may include various types of devices with input/output functions, such as a smart phone 130-1, a tablet computer 130-2, a laptop computer 130-3, a desktop computer 130-4, etc. In some embodiments, the user terminal 130 may provide a service interface, such as a graphical user interface.

In some embodiments, a user may send one or more of commands for starting/stopping scanning, scanning parameter configuration, data import/export, or the like, to the data acquisition device 110 through the user terminal 130.

In some embodiments, the user may set, through the user terminal 130, one or more parameters related to model training, such as a model type, a model structure, a count of channels of a model input/a model output, an iteration termination condition, or the like. In some embodiments, the user may define a task set through the user terminal 130. The task set may include a plurality of tasks. In some embodiments, the user may import a sample set through the user terminal 130. The sample set may include a plurality of samples. Each of the plurality of samples may include a model input (denoted as x) and a sample label (denoted as y).

In some embodiments, the user may obtain the predicted dose distribution information output by the dose distribution prediction model through the user terminal 130. In some embodiments, the user may access, modify, or copy the treatment plan of the subject through the user terminal 130.

The network 140 may be configured to connect components (e.g., the data acquisition device 110, the processing device 120, and the user terminal 130) of the system 100 to enable communication between the components. In some embodiments, the processing device 120 may obtain the relevant data of the subject from the data acquisition device 110 via the network 140 to realize dose prediction. In some embodiments, the processing device 120 may send the predicted dose distribution information output by the dose distribution prediction model to the user terminal 130 for access by the user via the network 140. In some embodiments, the user terminal 130 may send information and/or data input by the user, such as the parameters, the task set, the sample set, etc., related to model training to the processing device 120 via the network 140 for use by the processing device 120.

The network configured to connect the components of the system 100 may include a wired network and/or a wireless network. For example, the network 140 may include a cable network, a wired network, a fiber optic network, a telecommunications network, an intranet, an Internet, a local area network (LAN), a wide area network (WAN), a wireless local area network (WLAN), a metropolitan area network (MAN), a public switched telephone network (PSTN), a Bluetooth network, a zigbee network (ZigBee), near-field communication (NFC), an in-device bus, in-device wiring, cable connection, or the like, or any combination thereof. The network connection between two components may be implemented in one or more of the ways described above.

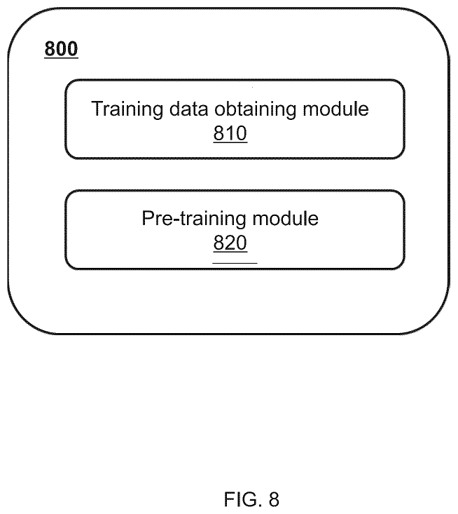

FIG. 2 is a flowchart illustrating an exemplary pre-training method for a dose distribution prediction model according to some embodiments of the present disclosure. In some embodiments, the process 200 may be stored in a storage medium as a form of instructions, and can be invoked and/or executed by a pre-training system 800 in FIG. 8. The operations of the illustrated process 200 presented below are intended to be illustrative. In some embodiments, the process 200 may be accomplished with one or more additional operations not described, and/or without one or more of the operations discussed. Additionally, the order in which the operations of the process 200 as illustrated in FIG. 2 and described below is not intended to be limiting. In some embodiments, the process 200 may be implemented in the system 100 (e.g., the processing device 120) that is provided at a hospital end, or other devices (e.g., provided at a supplier end) different from the system 100. As illustrated in FIG. 2, the process 200 may include the following operations.

In 210, for each of a plurality of training tasks, one or more samples corresponding to the training task may be obtained. In some embodiments, the operation 210 may be performed by a training data obtaining module 810. The training data obtaining module 810 may obtain a plurality of training tasks. For each of the plurality of training tasks, the training data obtaining module 810 may obtain one or more samples corresponding to the training task.

A task indicates a target for model training. A task may correspond to one or more sample. Each of the one or more sample corresponding to the task may include a model input and a label. The label may include labeled dose distribution information corresponding to the task. For example, a label of a sample corresponding to task Tᵢ may include labeled dose distribution information corresponding to the task Ti. The present disclosure does not specifically limit the manner of obtaining the label of a sample. Merely by way of example, the labeled dose distribution information may be dose distribution information determined using clinically validated simulation software based on a treatment plan, or actual dose distribution information (e.g., actually measured by any known or future technical means). When a task is used for pre-training, the task may be referred to as a training task. When a task is used for fine-tuning of a pre-training model, the task may be referred to as a target task. A task may correspond to one or more samples.

In some embodiments, a task may correspond to a subject or a group of subjects. For example, a first task may correspond to a subject or a group of subjects A, and a second task may correspond to a subject or a group of subjects B. A task corresponding to a subject a group of subjects may be understood as radiotherapy dose distribution prediction for the subject or the group of subjects. The full dose of radiation delivered to a subject or a group of subjects according to a treatment plan may be divided into a number (or count) of smaller doses referred to as fractions. The fractions as a series of treatment sessions may make up a radiotherapy course according to the treatment plan. The radiotherapy course may spread over a period of time, e.g., several weeks, several days. In some embodiments, each of the one or more samples of a task corresponding to a subject or a group of subjects may correspond to a treatment session of the subject or the group of subjects. For each of the one or more samples of a task corresponding to a subject or a group of subjects, a model input of the sample may include reference information (e.g., a subject image, which may include a lesion and/or a surrounding normal tissue) of the subject or a group of subjects of the corresponding treatment session, and a label of the sample may include labeled dose distribution information of the subject or the group of subjects (e.g., labeled dose distribution information of the lesion and/or the surrounding normal tissue in the subject image) of the corresponding treatment session.

In some embodiments, a task may correspond to a lesion type. Lesions corresponding to the same lesion type may have similarity in at least one of abnormality type, position, size, morphology, severity, contour, and treatment plan. For example, a first task may correspond to tumors in liver, and a second task may correspond to tumors in lungs. A task corresponding to a lesion type may be understood as radiotherapy dose distribution prediction for a specific lesion type. For each of the one or more samples of a task corresponding to a lesion type, a model input of the sample may include reference information (e.g., a subject image including a lesion of the lesion type) of a subject with a lesion of the lesion type, and a label of the sample may include labeled dose distribution information of the subject.

In some embodiments, a task may correspond to a body part (also referred to as a region of interest (ROI)) including a lesion. For example, a first task may correspond to lungs, a second task may correspond to a nasopharyngeal region, and a third task correspond to a cervical region. A task corresponding to a body part including a lesion may be understood as radiotherapy dose distribution prediction for the body part. For each of the one or more samples of a task corresponding to a body part, a model input of the sample may include an image of the body part, and a label of the sample may include labeled dose distribution information of the body part (e.g., the lungs) corresponding to the sample.

In some embodiments, a task may correspond to two or more body parts. The two or more body parts may have a plurality of lesions that require radiotherapy in the same fraction or treatment session (also referred to as simultaneous radiotherapy). For example, a first task may correspond to lungs and brain, and a second task may correspond to stomach, liver, and spleen. A task corresponding to two or more body parts may indicate that the two or more body parts have a plurality of lesions (e.g., a plurality of lesions in lungs and abdomen) that require simultaneous radiotherapy, and may be understood as radiotherapy dose distribution prediction for the two or more body parts. Since there are differences in the radiotherapy dose required for the two or more body parts, radiotherapy dose distribution prediction needs to be performed to each body part, i.e., a dose distribution prediction model may output predicted dose distribution information of each of the two or more body parts of the subject. For each of the one or more samples of a task corresponding to two or more body parts, a model input of the sample may include at least one image of each of the two or more body parts, and/or an image including the two or more body parts. A label of the sample may include labeled dose distribution information of each of the two or more body parts. For example, a task corresponds to lungs and brain. The model input of a sample corresponding to the task may include at least one lung image and at least one brain image, and/or an image including lungs and brain. The label of the sample corresponding to the task may include the labeled dose distribution information of the lungs and the brain. In some embodiments, images respectively corresponding to the two or more parts may correspond to the same subject or different subject. For example, for a sample of a task corresponding to lungs and brain, the model input of the sample may include a lung image of subject A and a brain image of subject B. As another example, for a sample of a task corresponding to lungs and brain, the model input of the sample may include a lung image and a brain image of subject C.

In some embodiments, an image including at least one body part of a subject corresponding to a sample may be acquired by scanning the at least one body part of the subject using a scanning device. In some embodiments, an image including at least one body part of a subject corresponding to a sample may be a reconstructed image, e.g., a multiplanar reconstruction (MPR) image, a curved planar reconstruction (CPR) image, a 3D render image, etc.

In some embodiments, when two or more body parts of a subject require simultaneous radiotherapy (e.g., when there is a plurality of lesions in the two or more body parts of the subject), the two or more body parts may interact with each other during radiotherapy, which in turn affects predicted dose distribution information of the two or more body parts. For example, assuming that the two or more body parts are close to each other, the dose distribution of the two or more body parts may be affected during a radiotherapy process. In addition, the two or more body parts having the plurality of lesions may have some correlation, e.g., there may be a rule in the predicted dose distribution information of the two or more body parts. Therefore, when dose distribution information is predicted for a task corresponding to two or more body parts, in order to make the predicted dose distribution information more accurate, it is necessary to consider the situation of the two or more body parts undergoing simultaneous radiotherapy.

An image including two or more body parts may reflect a positional relationship between the two or more body parts (e.g., a positional relationship between a plurality of lesions of the two or more body parts), which is conducive to dose distribution prediction of the two or more body parts. However, an image including two or more body parts with a plurality of lesions may be difficult to obtain. For example, it is difficult to encounter subjects who require simultaneous radiotherapy of two or more body parts, therefore it is difficult to encounter an existing image including two or more body parts including a plurality of lesions. In order to alleviate the problem of the count of samples, simulated images may be generated by image composition, thereby improving the effect of model training. In some embodiments, an image including two or more body parts may be obtained by: obtaining at least one sample image corresponding to each of the two or more body parts; and performing image composition based on the at least one sample image corresponding to each of the two or more body parts to determine the image including the two or more body parts. The sample image refers to a medical image including one or more body parts each of which includes at least one lesion. In some embodiments, the sample images corresponding to the two or more body parts used to obtain the composite image may correspond to the same subject or different subjects.

Image composition refers to a process of merging or fusing a plurality of sample images. In some embodiments, the manner of image composition may include image splicing, image fusion, etc. Merely by way of example, image composition may be realized by medical image processing software (e.g., ImageJ, MATLAB, etc.), a medical image fusion technique (e.g., an image fusion algorithm, image registration, etc.), etc.

It should be understood that a subject feature of a subject may also affect predicted dose distribution information and a radiotherapy plan. The model input of a sample corresponding to a task may further include a subject feature of a subject corresponding to the sample. The subject feature may include age, gender, height, weight, body dimension (e.g., waist dimension, thigh dimension, etc.), medical history, physiology indicator (e.g., blood pressure, heart rate, blood glucose, metabolism), or the like, or any combination thereof.

In some embodiments, a lesion feature of a lesion may also affect predicted dose distribution information and a radiotherapy plan. The model input of a sample corresponding to a task may further include a lesion feature of a lesion corresponding to the sample. The lesion feature of the lesion may include an abnormality type, a position, a size, morphology, a contour, severity, a treatment plan of the lesion, or the like, or any combination thereof.

In some embodiments, the model input of a sample of a task corresponding to at may further include feature information corresponding to the at least one body part.

The feature information may include a parameter reflecting a feature of the at least one body part. The feature information may include at least one of a type and a size of each of the at least one body part. In some embodiments, the feature information of a body part may include information of a foreign object inside the body part. The information of a foreign object may include a type, a position, a shape, a size, a material, or the like, of the foreign object. The foreign object may produce local dose elevation, dose scattering, dose occlusion, etc., which may also affect the predicted dose distribution information. For a task corresponding to two or more body parts, the feature information may further include at least one of a distance between the two or more body parts, a weight of each of the two or more body parts, and a simulated treatment device parameter.

The type of a body part refers to an organ or a tissue corresponding to the body part, such as lungs, brain, liver, cervix, etc. Different types of body parts have different tissue densities and compositions, which in turn affects the predicted dose distribution information.

The distance between two or more body parts refers to a spatial distance between the two or more body parts. The spatial distance between the two or more body parts may include a distance between geometric centers of the two or more body parts, centroids of the two or more body parts, or a minimum distance between edges of the two or more body parts. It should be understood that when the two or more body parts undergo simultaneous radiotherapy, the dose distribution of the two or more body parts may affect each other. For example, effects such as cross radiation, an adjacent organ effect, and an inter-tissue interaction may occur. The cross radiation refers to scattering of radiation rays during radiotherapy. The adjacent organ effect refers to that when radiation rays irradiate to one organ during radiotherapy, the radiation rays may have an effect on other organs surrounding the organ. Therefore, introducing the distance between the two or more body parts in the model input may reflect the influence of relative positions of the two or more body parts on the predicted dose distribution information of the two or more body parts and the radiotherapy plan.

In some embodiments, the diversity of samples corresponding to a task corresponding to two or more body parts may be increased by adjusting the distance between the two or more body parts. For example, a task corresponds to lungs and brain. The model input of a first sample corresponding to the task may include a lung image A, a brain image B, and a first distance between lungs and brain. The label of the first sample corresponding to the task may include first labeled dose distribution information of the lungs and the brain. By adjusting the first distance between lungs and brain in the first sample, a second sample corresponding to the task may be obtained. The model input of the second sample may include the lung image A, the brain image B, and a second distance between lungs and brain. The label of the second sample corresponding to the task may include second labeled dose distribution information of the lungs and the brain. As another example, a task corresponds to lungs and brain. Lung image A and brain image B may be used to generate a first composite image that indicates a first distance between lungs and brain. The model input of a first sample corresponding to the task may include the first composite image and the first distance between lungs and brain. The label of the first sample corresponding to the task may include first labeled dose distribution information of the lungs and the brain. By adjusting the first distance between lungs and brain, a second composite image may be generated by performing image composition on the lung image A and the brain image B based on a second distance lungs and brain. The model input of a second sample corresponding to the task may include the second composite image and the second distance between lungs and brain. The label of the second sample corresponding to the task may include second labeled dose distribution information of the lungs and the brain.

a second sample corresponding to the task may be obtained. The model input of the second sample may include the lung image A, the brain image B, and a second distance between lungs and brain. The label of the second sample corresponding to the task may include second labeled dose distribution information of the lungs and the brain.

The weight of each of the two or more body parts refers to an importance of each of the two or more body parts in the radiation treatment. In some embodiments, under the influence of factors such as severity of the lesions of the two or more body parts, a treatment order of the two or more body parts in a treatment session, and sensitivity of the two or more body parts to radiation, the treatment device 120 may set a weight for each of the two or more body parts to reflect the influence of the above factors on the predicted dose distribution information. In some embodiments, for a body part, the more severe the lesion, the greater the weight corresponding to the body part. In some embodiments, for a body part, the more advanced the treatment order, the greater the weight corresponding to the body part. In some embodiments, for a body part, the greater the sensitivity to radiation, the greater the weight corresponding to the body part. In some embodiments, the weight of each of the two or more body parts may also be a weight for image composition. For example, a sample image A corresponds to lungs and a sample image B corresponds to brain. An image C including lungs and brain may be obtained by performing image composition on the sample image A and the sample image B. The image C may be obtained by Equation (1):

$$C = {w}_{a} * A + {w}_{b} * B , \qquad ( 1 )$$

$${w}_{a} + {w}_{b} = 1 , \qquad ( 2 )$$

wherein C denotes the image C, wₐ denotes a weight corresponding to lungs, and w_{b} denotes a weight corresponding to brain. By adjusting the weight of a body part, a degree of contribution of the body part in image composition may be controlled. Merely by way of example, the greater the weight corresponding to the body part, the greater the contrast of the body part in the composite image, and the more easier the body part is recognized in the composite image.

The simulated treatment device parameter refers to a virtual parameter for performing radiotherapy to each of the two or more body parts set based on parameter information of an actual radiotherapy treatment device. In order to make the treatment device cover as many body parts as possible in the task, parameters of the treatment device may be adjusted to meet the needs of use. The simulated treatment device parameter may be used as the input of the model to better guide predicting the dose distribution and the subsequent radiotherapy plan. Merely by way of example, the simulated treatment device parameter may include an energy range of a radiation source, an angle of the radiation source, or the like.

In some embodiments, for a task corresponding to two or more body parts, the feature information may include a position of each of the two or more body parts in a scanning device. For example, for a task corresponding to lungs and brain, the model input of a sample of the task may include a lung image and a brain image. The feature information may include a position of lungs in a first scanning device when the lungs are scanned by the first scanning device to obtained the lung image, and a position of brain in a second scanning device when the brain are scanned by the second scanning device to obtained the brain image. As another example, for a task corresponding to lungs and brain, the model input of a sample of the task may include a composite image including lungs and brain. The composite image may be obtained by performing image composition on a lung image and a brain image. The feature information may include a position of lungs in a first scanning device when the lungs are scanned by the first scanning device to obtained the lung image, and a position of brain in a second scanning device when the brain are scanned by the second scanning device to obtained the brain image.

For image composition, or the images (used as the model input) respectively corresponding to the two or more body parts, image registration may be required for the images corresponding to the two or more body parts to ensure that the images are aligned under a same coordinate system. Image registration may be performed in a better way by considering the position of each of the two or more body parts in a scanning device during the pre-training.

In some embodiments, image registration may be first performed on the images corresponding to the two or more body parts based on the position of each of the two or more body parts in a scanning device. Then, the images after registration may be used as the model input or may be used to obtain the composite image.

In some embodiments, a task may correspond to a weight combination. The weight combination may include a Planning Target Volume (PTV) weight and an Organ At Risk (OAR) weight. The PTV refers to an expanded irradiation region in a radiotherapy plan for considering a movement of an organ, and a change in a target position and a target volume during irradiation. The OAR refers to an organ that needs to be protected around the PTV. The PTV weight indicates a dose requirement for the PTV, and the OAR weight indicates a dose requirement for the OAR. The units for the both weights may be in radiation dose Gy. a task corresponding to a weight combination may be understood as radiotherapy dose distribution prediction based on a dose requirement for a PTV and a dose requirement for an OAR. For each of the one or more samples of a task corresponding to a weight combination, a model input of the sample may include the weight combination, and a label of the sample may include labeled dose distribution information that meets a dose requirement corresponding to the weight combination.

Depending on different radiotherapy cases or different styles of physicians, trade-offs of physicians in dose coverage of a target region and a dose limit of OAR during treatment may be different. Some radiotherapy cases (or physicians) may place more emphasis on the dose coverage of the target region, while some radiotherapy cases (or physicians) may place more emphasis on the dose limit of OAR. Therefore, predicting only one dose distribution is difficult to meet the diverse treatment needs of the physicians. Therefore, the present disclosure provides a multi-target dose prediction model. By incorporating the technique of meta-learning, the multi-target dose prediction model provided by the present disclosure is capable of predicting 3D dose distribution corresponding to an input PTV weight and an input OAR weight.

It should be understood that the present disclosure does not specifically limit the definition of tasks. Referring to the foregoing embodiments, a task may also correspond to any combination of a subject, a lesion type, at least one body part, and a weight combination (a dose requirement). For example, a task correspond to a lesion type, a body part, and a weight combination may be understood as radiotherapy dose distribution prediction for the body part with a lesion of the lesion type according to the weight combination.

In some embodiments, the plurality of training tasks in operation 210 may be selected from a task set defined by a user (e.g., a doctor, an engineer, a technician, a supplier, etc.). The plurality of training tasks may include at least one task each of which corresponds to a subject. In some embodiments, the plurality of training tasks may include at least one task each of which corresponds to a lesion type. In some embodiments, the plurality of training tasks may include at least one task each of which corresponds to at least one body part. In some embodiments, the plurality of training tasks may include at least one task each of which corresponds to a weight combination. In some embodiments, the plurality of training tasks may include at least one task each of which corresponds to any combination of a subject or a group of subjects, a lesion type, at least one body part, and a weight combination.

In 220, a pre-training model may be obtained based on the samples corresponding to the plurality of training tasks. In some embodiments, the operation 220 may be performed by a pre-training module 820.

For example, the pre-training model may be obtained by various methods such as a meta-learning method, a migration learning method, etc. The pre-training model may be adjusted based on one or more samples under a target task to obtain a dose distribution prediction model for the target task. Merely by way of example, the objective of meta-learning is to learn a set of “good” pre-training parameters, which are parameters of the pre-training model. Since the plurality of training tasks include various types of tasks, the pre-training model obtained using the plurality of training tasks may be a general model having experience of dose distribution prediction for various types of tasks, so that the pre-training model can converge quickly on a small count of samples (even a single sample) corresponding to a target task to obtain a dose distribution prediction model for the target task.

More details regarding the pre-training phase may be found in FIG. 3 and related descriptions thereof.

FIG. 3 is a flowchart illustrating an exemplary pre-training phase according to some embodiments of the present disclosure. As illustrated in FIG. 3, a process 300 may include the following operations.

In 310, model parameters may be initialized.

In 320, a group of tasks may be extracted, as training tasks, from a task set.

In some embodiments, the task set may be divided into a plurality of subsets. At least one task may be randomly extracted from at least one of the plurality of subsets, so that the group of tasks are extracted. In some embodiments, at least one task may be extracted from the task set at a time, and after a plurality of times, the group of tasks are extracted. In some embodiments, the group of tasks may be extracted from the task set in one time.

The difference between a conventional machine learning method with meta-learning is that the meta-learning treats one or more samples corresponding to a task as a single piece of training data (used in an iteration), while the conventional machine learning method treats a sample as a single piece of training data. Thus, when the model parameters are adjusted using batch stochastic gradient descent, the group of tasks extracted in the operation 320 may correspond to a batch of training data (a plurality of pieces of training data).

For ease of description, the group of tasks may be denoted as { [symbol] ₁, [symbol] ₂, . . . , [symbol] _{M}}, wherein an extracted task in the group of tasks may be denoted as [symbol] ₁, i=1, 2, . . . , M. The tasks in the task set obey a distribution p( [symbol] ), and [symbol] ᵢ-p( [symbol] ).

For task [symbol] ᵢ in the group of tasks, the pre-training module 820 may perform a first gradient determination and a first parameter update, i.e., operations 330-340.

In 330, one or more first samples may be extracted from a support set of task [symbol] ᵢ for the first gradient determination.

A gradient of the first gradient determination may be denoted as ∇_{θ} [symbol] (f_{θ}), wherein θ denotes the initialized model parameters in operation 310 (f_{θ} denotes a model corresponding to the initialized model parameters), and [symbol] denotes a loss function.

[symbol]

In some embodiments, for a first sample of task [symbol] ᵢ, the model input of the first sample may be input into the model f_{θ}, and the model f_{θ} may output a first sample dose distribution information. A loss function value may be determined based on the first sample dose distribution information and the label of the first sample. When two or more first samples are extracted from the support set of task [symbol] ᵢ for the first gradient determination, a sum of the loss function values corresponding to the two or more first samples may be determined as a first loss function value corresponding to task [symbol] ᵢ. The gradient of the first gradient determination corresponding to task [symbol] ᵢ may be determined based on the first loss function value corresponding to task [symbol] ᵢ.

In the pre-training phase, a sample set for each task may be divided into a support set and a query set. The support set may be used for the first gradient determination, and the query set may be used for a second gradient determination.

In 340, the first parameter update may be performed based on the gradient of the first gradient determination.

The first parameter update may be denoted as θ′ᵢ=θ−α∇_{θ} [symbol] (f_{θ}), wherein θ*ᵢ denotes model parameters after the first parameter update, θ denotes model parameters before the first parameter update (e.g., the initialized model parameters in operation 310), and a denotes a learning rate of the first parameter update.

In some embodiments, operations 330 and 340 may be performed for each task in the group of tasks.

In 350, for each task of the group of tasks, one or more second samples may be extracted from a query set of the task to perform a second gradient determination.

The result θ′ᵢ of the first parameter update corresponding to each task in the group of tasks may be used for the second gradient determination, and a gradient of the second calculation may be denoted as ∇_{θ}Σ [symbol] ᵢ(f_{θ′}_{ᵢ}). In some embodiments, for a second sample of task [symbol] ᵢ, the model input of the second sample may be input into the model f_{θ′}_{ᵢ}, and the model f_{θ′}_{ᵢ} may output a second sample dose distribution information. A loss function value may be determined based on the second sample dose distribution information and the label of the second sample. When two or more second samples are extracted from the query set of task [symbol] for the second gradient determination, a sum of the loss function values corresponding to the two or more second samples may be determined as a second loss function value corresponding to task [symbol] . The gradient of the second gradient determination may be determined based on the second loss function value corresponding to task [symbol] . It can be seen that the gradient of the first gradient determination may be determined based on the loss function value under a single task, and the gradient of the second gradient determination may be determined based on a sum of loss function values of the group of tasks.

In 360, a second parameter update may be performed based on the gradient of the second gradient determination.

The second parameter update may be denoted as θ←θ−β∇_{θ} [symbol] [symbol] f_{θ′}_{ᵢ}), wherein β denotes a learning rate of the second parameter update. It should be noted that a starting point of the second parameter update is the model parameters θ before the first parameter update (e.g., the initialized model parameters in operation 310), i.e., the second parameter update is to update the initialized model parameters in operation 310.

In some embodiments, the process 300 may be executed iteratively until a termination condition is satisfied. That is, the process 300 may be an iteration of the pre-training process. The model parameters finally obtained in each iteration (the model parameters obtained after the second gradient determination update in the operation 360) may be used as initialized model parameters for a next iteration. The model parameters finally obtained in the last iteration may be used to construct the pre-training model. The terminal condition refers to that a count of iterations that have been performed reaches an expectation, a change in the model parameters (e.g., a change between the initialized model parameters in operation 310 and the updated model parameters obtained after the second parameter update in operation 360) is less than a threshold, the gradient of the second gradient determination is less than a threshold, a change between the gradient of the second gradient determination in the current iteration and the gradient of the second gradient determination in the previous iteration is less than a threshold, etc. In some embodiments, groups of tasks of a plurality of iterations may form the plurality of training tasks in operation 210.

In the first iteration, in operation 310, the model parameters may be randomly initialized or set, or may be set as a specific value, e.g., 1.

The loss function in the process 300 may be set based on a nature of the model. For example, for a regression model, the loss function may be constructed based on a mean squared error (MSE). As another example, for a classification model, the loss function may be constructed based on a cross entropy.

Merely by way of example, for a generative adversarial network, the loss function may be determined as follows:

$$ℒ = {ℒ}_{a d v} + {ℒ}_{m s e} , \qquad ( 3 )$$

$${ℒ}_{a d v} = {E}_{x , y} [ log D ( x , y ) ] + {E}_{x} [ log ( 1 - D ( x , G ( x ) ) ) ] , \qquad ( 4 )$$

$${ℒ}_{m s e} = {E}_{x , y} [ {‖ y - G ( x ) ‖}_{2} ] , \qquad ( 5 )$$

wherein [symbol] denotes the loss function, [symbol] _{adv} denotes a generative adversarial loss, and [symbol] ₘₛₑ denotes an MSE loss; G denotes a generator, D denotes a discriminator, x denotes a model input of the generative adversarial network, and y denotes a sample label (i.e., labeled dose distribution information); and E denotes expectation, log denotes a logarithmic function, and ∥ ∥₂ denotes a 2-norm.

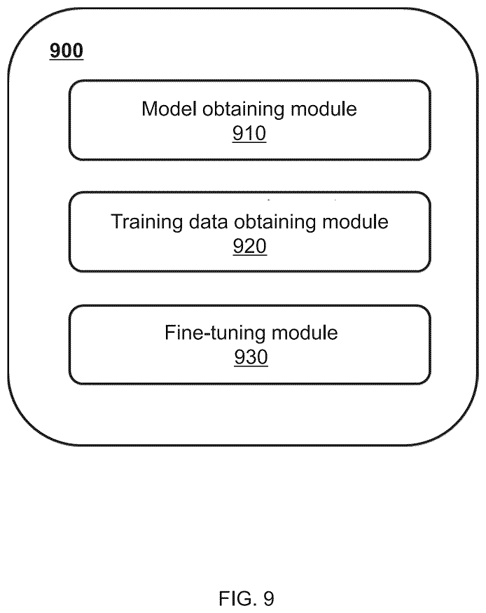

FIG. 4 is a flowchart illustrating an exemplary training method for a dose distribution prediction model according to some embodiments of the present disclosure. In some embodiments, the process 400 may be stored in a storage medium as a form of instructions, and can be invoked and/or executed by a training system 900 in FIG. 9. The operations of the illustrated process 400 presented below are intended to be illustrative. In some embodiments, the process 400 may be accomplished with one or more additional operations not described, and/or without one or more of the operations discussed. Additionally, the order in which the operations of the process 400 as illustrated in FIG. 4 and described below is not intended to be limiting. In some embodiments, the process 400 may be implemented in the system 100 (e.g., the processing device 120) that is provided at a hospital end. As illustrated in FIG. 4, the process 400 may include the following operations.

In 410, a pre-training model may be obtained. In some embodiments, the operation 410 may be performed by a model obtaining module 910.

The pre-training model may be obtained by various methods such as a meta-learning method, a migration learning method, etc. More descriptions the pre-training model and meta-learning may be found in the related descriptions of the process 200 and the process 300.

In some embodiments, a pre-training end (e.g., a software service provider) may train a pre-training model in advance and distribute the pre-training model to one or more application ends (e.g., hospitals). The application end may fine-tune the pre-training model based on a target task to obtain a dose distribution prediction model for the target task.

In 420, one or more samples under a target task may be obtained. In some embodiments, the operation 420 may be performed by a training data obtaining module 920. The target task may aim for a target subject to be treated (e.g., using the treatment device in the system 100).

During the fine-tuning phase, a sample set of the target task may also be divided into a support set and a query set. The support set may be used for fine-tuning (i.e., operation 430) of model parameters of the pre-training model, and the query set may be used to test a prediction accuracy of the model after fine-tuning (i.e., a dose distribution prediction model) on the target task.

In some embodiments, the one or more samples of the target task may be obtained based on a historical treatment plan of the target subject. For example, the target task may be does distribution prediction for lungs of the target subject. The target subject has received a radiotherapy on lungs. A lung image including a lesion of the target subject and actual dose distribution information in the radiotherapy may be used as a sample of the target task. When there is no data of the target subject to be used as a sample of the target task, e.g., the target subject does not have a historical treatment plan for lungs, resulting in unavailability of actual dose distribution information as sample labels, the training data obtaining module 920 may determine treatment data of a candidate subject whose similarity to the target task of the target subject satisfies a preset condition as a sample of the target task. Merely by way of example, the target task may be does distribution prediction (meeting a weight combination) for lungs of the target subject. The preset condition may include that a similarity between a lung image of the candidate subject and a lung image of the target subject exceeds a first similarity threshold (e.g., 90%) and a similarity between a dose requirement (e.g., a weight combination of a PTV weight and an OAR weight) of the target subject and a dose requirement for the candidate subject exceeds a second similarity threshold (e.g., 90%). The lung image of the candidate subject and actual dose distribution information corresponding to the lung image of the candidate subject may be used as a sample of the target task.

In 430, the pre-training model may be adjusted based on the one or more samples under the target task to obtain a dose distribution prediction model for the target task. In some embodiments, the operation 430 may be performed by a fine-tuning module 930.

Since the pre-training model has good generalization capacity, the pre-training model may be quickly adapted to different tasks with a small count of samples of the target task to obtain the dose distribution prediction model for the target task.

In some embodiments, the fine-tuning module 930 may determine a first gradient ∇_{θ} [symbol] (f_{θ}_{ₚᵣₑ}) based on one or more samples extracted from the support set of the target task, wherein [symbol] refers to the target task, θₚᵣₑ refers to the model parameters of the pre-training model, and f_{θ}_{ₚᵣₑ} refers to the pre-training model. For a sample extracted from the support set of the target task, the model input of the sample may be input into the pre-training model f_{θ}_{ₚᵣₑ}, and the pre-training model f_{θ}_{ₚᵣₑ} may output a third sample dose distribution information. A loss function value may be determined based on the third sample dose distribution information and the label of the sample. When two or more samples are extracted from the support set of the target task, a sum of the loss function values corresponding to the two or more samples may be used to determine the first gradient. A parameter update may be performed on the pre-training model based on the first gradient to obtain the dose distribution prediction model for the target task.

In some embodiments, the fine-tuning module 930 may determine a second gradient ∇_{θ} [symbol] (f_{θ}_{ₜ}) based on one or more samples extracted from the query set of the target task, wherein θₜ refers to the model parameters of the dose distribution prediction model, and f_{θ}_{ₜ} refers to the dose distribution prediction model. For a sample extracted from the query set of the target task, the model input of the sample may be input into the dose distribution prediction model f_{θ}_{ₜ}, and the dose distribution prediction model f_{θ}_{ₜ} may output a fourth sample dose distribution information. A loss function value may be determined based on the fourth sample dose distribution information and the label of the sample. When two or more samples are extracted from the query set of the target task, a sum of the loss function values corresponding to the two or more samples may be used to determine the second gradient. The fine-tuning module 930 may determine whether the second gradient is less than a gradient threshold. In response to determining that the second gradient is less than the gradient threshold, the fine-tuning module 930 may output the dose distribution prediction model for the target task. In response to determining that the second gradient is larger than or equal to the gradient threshold, the fine-tuning module 930 may perform a parameter update on the dose distribution prediction model based on the second gradient, and extract one or more samples from the query set of the target task to test the accuracy of the updated dose distribution prediction model, until the gradient is less than the gradient threshold.

In some embodiments, under a meta-learning framework, a dose distribution prediction model for a single task can be obtained by meta-learning. Specifically, assuming that there are N tasks, denoted as T₁-T_{N}, for task Tᵢ (1≤i≤N), a dose distribution prediction model (denoted as Mᵢ) for the task Tᵢ may be obtained through meta-learning. In some embodiments, the dose distribution prediction model may also be obtained by migration learning. The dose distribution prediction model for a task may output predicted dose distribution information corresponding to the task. For example, the dose distribution prediction model Mᵢ for the task Tᵢ may output the predicted dose distribution information corresponding to the task Tᵢ.

In some embodiments, an input of the dose distribution prediction model may include one or more of an image of at least one body part (a ROI, e.g., lungs) of the target subject, contour information (or outlining information) of a PTV, contour information of an OAR, a dose requirement for the PTV (e.g., a PTV weight), and a dose requirement for the OAR (e.g., an OAR weight). The contour information of the PTV/OAR indicates a contour of the PTV/OAR. Specifically, the contour information of the PTV/OAR may be represented as a segmented image of the PTV/OAR, which may be obtained by segmenting from the image of the ROI (e.g., the lungs). More descriptions regarding the input of the pre-training model and the model input for the one or more samples may be found in the embodiments above. An output of the dose distribution prediction model for the target task may include predicted dose distribution information under the target task. The predicted dose distribution information may be configured to generate a new treatment plan for the target subject.

In some embodiments, the processing device 120 may also adjust the pre-trained model based on the one or more samples under the target task using multitask learning to obtain a treatment prediction model for the target task.

The treatment prediction model refers to a model configured to predict a treatment result and/or a risk of complications. In some embodiments, an output of the treatment prediction model may include a predicted treatment result and/or a risk of complications. Since there is a certain medical correlation (different predicted dose distribution information leads to changes in the predicted treatment result and the risk of complications) in the predicted dose distribution information, the predicted treatment result, and the risk of complications. Further analysis of the radiotherapy process may be realized by multitask learning. Merely by way of example, multitask learning may include joint learning, sequential learning, hierarchical learning, cooperative learning, etc. In some embodiments, an input of the treatment prediction model may be the same as the input of the dose distribution prediction model. For example, there may be one input layer and a plurality of output layers in a model, e.g., a first output layer for outputting predicted does distribution, and a second output layer for outputting a predicted treatment result. By sharing underlying representation, the model may learn common features in the input data, while the separate output layers may allow the model to remain flexibility across different tasks. In some embodiments, the treatment prediction model and the dose distribution prediction model may be the same model. For example, the predicted dose distribution information, the predicted treatment result, and the risk of complications may be simultaneously output by the model. The present disclosure does not limit the content of the task of multitask learning. Merely by way of example, the model may also output information related to a treatment schedule, a cost of the treatment, a need for a treatment instrument, etc.

In some embodiments, the processing device 120 may set a weight for each of a plurality of tasks in multitask learning based on user needs. For example, the processing device 120 may balance the importance of different tasks by adjusting the weights of different tasks in the loss function based on the degree of need for output information of each type of tasks set by the user. The weight may be determined by user settings.

In some embodiments, overfitting of the model can be mitigated through multitask learning, the generalization capability of the model can be improved, and the utilization efficiency of the training samples can be improved. The dose distribution prediction and the treatment result prediction may have correlation, which is conducive to optimizing the model, and improving the overall performance and the training efficiency. In addition, the model may predict the treatment result, which is conducive to physician planning of subsequent treatment in advance.

It should be noted that the principles of the present disclosure are applicable to any machine learning model, e.g., a linear regression model, a logistic regression models, a vector support machine, a decision tree, a random forest, a Bayesian model, a neural network (e.g., a deep learning model), etc.

In some embodiments, the pre-training model may include a conditional generative adversarial network. The generative adversarial network may include a generator and a discriminator. The conditional generative adversarial network may also include conditional information in an input compared to common generative adversarial networks. For the dose prediction problem, the input of the conditional generative adversarial network (i.e., an input of the generator) may include an image of at least one body part, contour information of a PTV, contour information of an OAR, a PTV weight, and an OAR weight, and an output of the generative adversarial network (i.e., an output of the generator) may include the predicted dose distribution information. It should be noted that in a prediction scenario, the dose distribution prediction model may retain only the generator in the (conditional) generative adversarial network or mask the discriminator. In addition, the pre-training model may also include other pre-training models, such as an end-to-end convolutional neural network (e.g., ResNet, DenseNet, U-Net, etc.), a Bayesian neural network, etc.

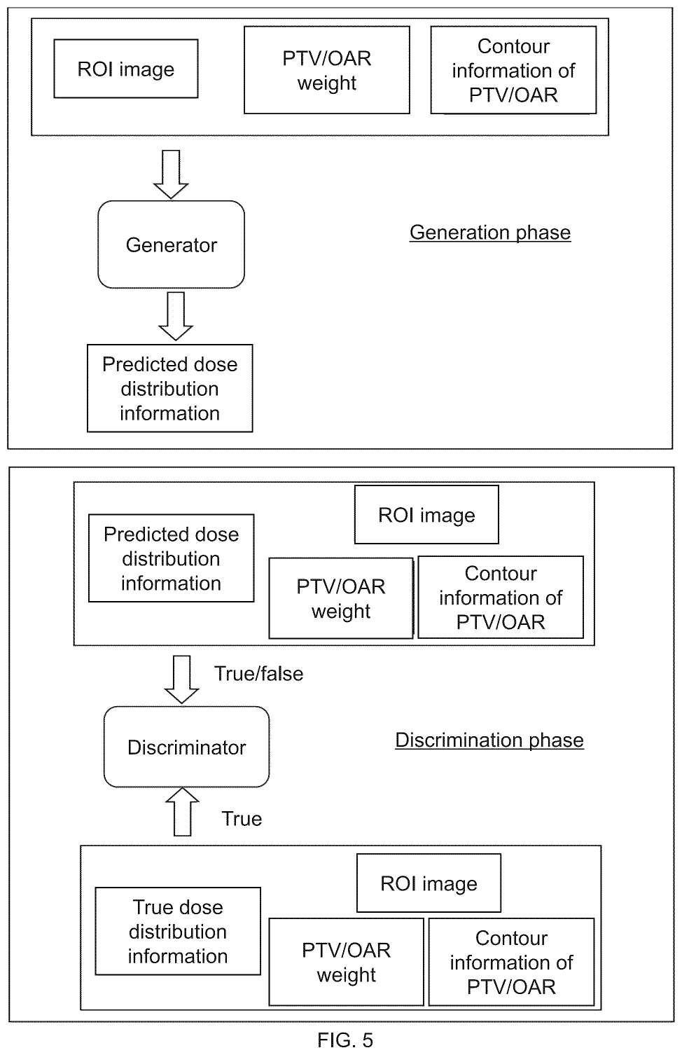

FIG. 5 is a schematic diagram illustrating a data flow of a conditional generative adversarial network according to some embodiments of the present disclosure.

As illustrated in FIG. 5, the data flow may be divided into a generation phase and a discrimination phase. In the generation phase, an image of at least one body part (an ROI image), a PTV/OAR weight, and PTV/OAR contour information may be sent to the generator. The generator may be guided by these inputs to generate predicted dose distribution information. In the discrimination phase, on the one hand, the image of the at least one body part, the PTV/OAR weight, the PTV/OAR contour information, and the generated predicted dose distribution information may be sent to the discriminator as negative samples, and on the other hand, the image of the at least one body part, the PTV/OAR weight, the PTV/OAR contour information, and labeled dose distribution information may be sent to the discriminator as positive samples. During a training process, parameters of the generator may be continuously optimized to generate dose distribution information that tends to be true (the labeled dose distribution information may be regarded as the true dose distribution information) to deceive the discriminator, and parameters of the discriminator may be continuously optimized to differentiate between the labeled dose distribution information and the generated predicted dose distribution information under defined conditions. Ultimately, the generator may generate the predicted dose distribution information close to the labeled dose distribution information, and the discriminator may accurately differentiate between the labeled dose distribution information and the generated predicted dose distribution information.

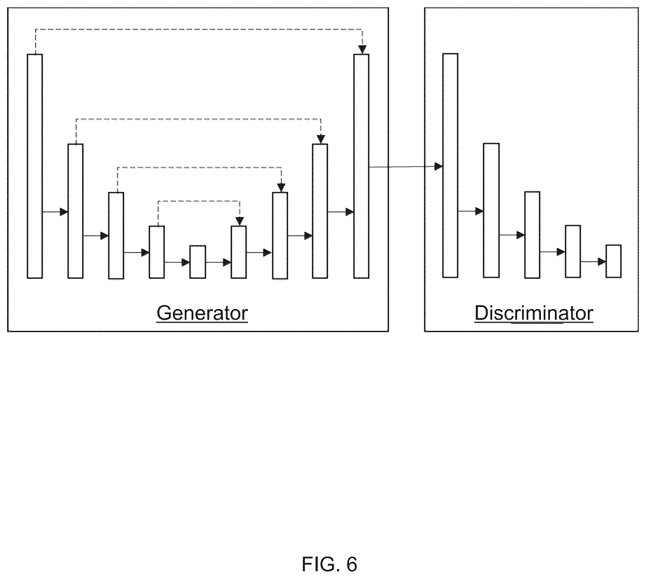

FIG. 6 is a schematic diagram illustrating a structure of a conditional generative adversarial network according to some embodiments of the present disclosure.

Referring to FIG. 6, a generator in the conditional generative adversarial network may include a downsampling part and an upsampling part. Rectangular bars in the figure represent image features. The longer the rectangular bars, the larger the sizes of the image features. The sizes of the image features decrease in a downsampling (solid arrows) process. The sizes of the image features increase in an upsampling (solid arrows) process. Downsampling times and upsampling times are both 4 times in the generator, as illustrated in FIG. 6. Correspondingly, a discriminator in the conditional generative adversarial network may also have 4 downsampling times as illustrated in FIG. 6.

In some embodiments, the generator in the conditional generative adversarial network may further include a feature splicing part. The image features in the downsampling process may be spliced with the image features in the upsampling process on a layer-by-layer basis. For example, as illustrated in FIG. 6, the image features before first downsampling may be spliced with features (dotted arrows) after fourth upsampling, the image features before second downsampling may be spliced with features (dotted arrows) after third upsampling, the image features before third downsampling may be spliced with features (dotted arrows) after second upsampling, and the image features before fourth downsampling features may be spliced with features (dotted arrows) after first upsampling. Details lost in the downsampling process, such as shallow features, edge features, etc., can be preserved through feature splicing, which in turn can improve the accuracy of dose prediction.

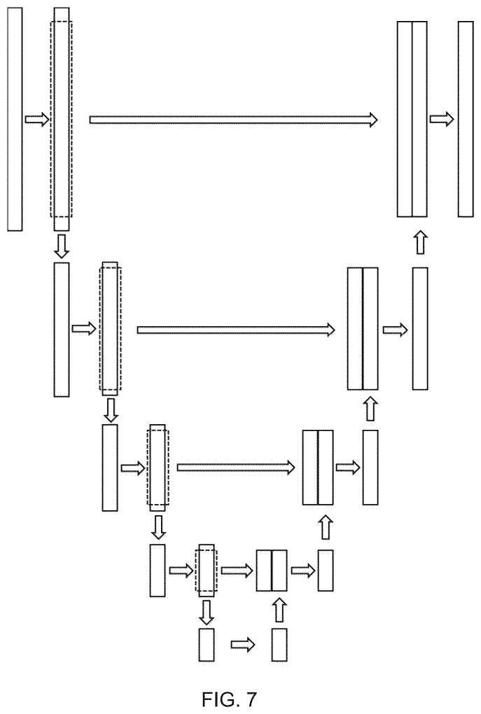

FIG. 7 is a schematic diagram illustrating a structure of a generator according to some embodiments of the present disclosure.

As illustrated in FIG. 7, the generator may adopt a U-Net structure consisting of an encoder (or an encoding part) and a decoder (or a decoding part). An encoding process may include multiple convolutions and multiple (e.g., 4) downsampling times. First, after preset times of (e.g., 2) convolutions (horizontal arrows) are performed, first downsampling may be performed (downward arrows). Then after each downsampling is performed, the preset times (e.g., 2) of convolutions may be performed. A decoding process may include multiple (e.g., 4) upsampling times and multiple convolutions. After each upsampling (upward arrow) is performed, the preset times (e.g., 2) of convolutions (horizontal arrows) may be performed. The decoder may receive features from each layer of the encoder in a cascade in addition to high-dimensional features output from the encoder. Specifically, features output from a kth layer in the encoder (counting from top to bottom in the figure, e.g., a first layer) may be cropped (referring to FIG. 7, a part in a dotted box may be retained) and then spliced with features in the decoder sent to an (N+1−k)th layer (counting from bottom to top in the figure, e.g., a fourth layer), and a splicing result may serve as an input for a convolution operation on the layer. In some embodiments, a processing logic of the discriminator may also include N times of downsampling (not shown in the figure), i.e., the generator and the discriminator may have the same times of downsampling.

It should be understood that the model structure in FIG. 6 and FIG. 7 is for example and illustration purposes only. For example, the number of times of sampling may also be set to other values, such as N=3 or 5.

In some embodiments, each convolution may be implemented by a 3D convolutional layer and an immediately following Rectified Linear Unit (ReLU function). Downsampling may be performed using a pooling operation (e.g., maximum pooling), and upsampling may be performed using an inverse convolution operation.

Merely by way of example, an input of the encoder may include a tensor of a size (shape) B×C×Z×H×W, wherein B denotes a batch size (i.e., a count of tasks contained in the group of tasks extracted in the operation 320), C denotes a count of channels, e.g., C may be 3, and the 3 channels may represent an image of ROI, contour information of a PTV, and contour information of an OAR, respectively, and Z, H, and W denote a depth, a height, and a width of the ROI, respectively. To ensure the consistency of samples, Z, H, and W may all be set to 128. An output of the generator may include a map of predicted dose distribution with a size (shape) of B×1×Z×H×W (e.g., 2×1×128×128×128). An input of the discriminator may include a tensor of a size (shape) B×(C+1)×Z×H×W (e.g., 2×4×128×128×128). Ultimately, the discriminator may output 0 or 1 to indicate whether the input map of dose distribution is a map of generated predicted dose distribution or a map of true dose distribution.

It should be noted that the foregoing description of the process is intended to be exemplary and illustrative only and does not limit the scope of application of the present disclosure. For a person skilled in the art, various corrections and changes to the process can be made under the guidance of the present disclosure. However, these corrections and changes remain within the scope of the present disclosure.

FIG. 8 is a block diagram illustrating an exemplary pre-training system for a dose distribution prediction model according to some embodiments of the present disclosure. In some embodiments, a pre-training system 800 may be implemented on a device (e.g., the processing device 120, or a processing device provided at the software service provider) configured to obtain the pre-training model.

As illustrated in FIG. 8, the pre-training system 800 may include the training data obtaining module 810 and the pre-training module 820.

The training data obtaining module 810 may be configured to obtain, for each of a plurality of training tasks, one or more samples corresponding to the training task.

The pre-training module 820 may be configured to obtain a pre-training model based on the samples under the plurality of training tasks. The pre-training model may be configured to be adjusted based on one or more samples under a target task to obtain a dose distribution prediction model for the target task. The dose distribution prediction model for the target task may be configured to output predicted dose distribution information under the target task.

More details regarding the pre-training system 800 and modules thereof may be found in FIG. 2 and FIG. 3 and related descriptions thereof.

FIG. 9 is a block diagram illustrating an exemplary training system for a dose distribution prediction model according to some embodiments of the present disclosure. In some embodiments, a training system 900 may be implemented on the processing device 120.

As illustrated in FIG. 9, the training system 900 may include the model obtaining module 910, the training data obtaining module 920, and the fine-tuning module 930.

The model obtaining module 910 may be configured to obtain a pre-training model.

The training data obtaining module 920 may be configured to obtain one or more samples under a target task. Labels of the one or more samples under the target task may include labeled dose distribution information under the target task.

The fine-tuning module 930 may be configured to adjust the pre-training model based on the one or more samples under the target task to obtain a dose distribution prediction model for the target task. The dose distribution prediction model for the target task may output predicted dose distribution information under the target task.

More details regarding the training system 900 and modules thereof may be found in FIG. 4 and related descriptions thereof.

It should be understood that the system and the modules thereof in FIG. 8 and FIG. 9 can be implemented in various ways. For example, in some embodiments, the system and the modules thereof may be implemented by hardware, software, or a combination of software and hardware. The hardware portion may be implemented utilizing a specialized logic; and the software portion may be stored in memory and executed by an appropriate instruction execution system, such as a microprocessor or specially designed hardware. It should be understood that for those skilled in the art, the methods and systems described above may be implemented using computer executable instructions and/or included in processor control code, such as provided on carrier media such as disks, CDs or DVD-ROMs, programmable memory such as read-only memories (firmware), or data carriers such as optical or electronic signal carriers. The system and the modules of the present disclosure may be implemented not only with hardware circuits such as ultra-large scale integrated circuits or gate arrays, semiconductors such as logic chips, transistors, etc., or programmable hardware devices such as field-programmable gate arrays, programmable logic devices, etc., but also with software executed, for example, by various types of processors, or with a combination (e.g., firmware) of the above hardware circuits and software.

It should be noted that the above description of the system and the modules thereof is for ease of description only, and does not limit the present disclosure to the scope of the cited embodiments. It is understood that for a person skilled in the art, with an understanding of the principle of the system, it may be possible to arbitrarily combine modules or form subsystems to be connected to other modules without departing from this principle. For example, in some embodiments, the model obtaining module 910 and the training data obtaining module 920 may be different modules in a single system, or a single module implementing the functionality of both modules. As another example, in some embodiments, the pre-training system 800 and the training system 900 may be two systems or may be combined into one system. Such variations are within the scope of protection of the present disclosure.

Beneficial effects of the embodiments of the present disclosure include, but are not limited to the following contents. (1) The model can be quickly adapted to new tasks (e.g., new subjects and new parts) by fine-tuning through a small count of samples or even a single sample based on the meta-learning method, and multi-target dose prediction can be realized under the small count of samples. (2) Inside the generative adversarial network, the features of the various layers of the encoder are retained by cascading to improve the model accuracy. It should be noted that the beneficial effects of different embodiments may be different, and the beneficial effects of different embodiments may be any one or a combination thereof, or any other beneficial effect that may be obtained.

The basic concept has been described above. Obviously, for those skilled in the art, the above detailed disclosure is only an example, and does not constitute a limitation to the present disclosure. Although not expressly stated here, those skilled in the art may make various modifications, improvements and corrections to the present disclosure. Such modifications, improvements and corrections are suggested in this disclosure, so such modifications, improvements and corrections still belong to the spirit and scope of the exemplary embodiments of the present disclosure.

Meanwhile, the present disclosure uses specific words to describe the embodiments of the present disclosure. For example, “one embodiment”, “an embodiment”, and/or “some embodiments” refer to a certain feature, structure or characteristic related to at least one embodiment of the present disclosure. Therefore, it should be emphasized and noted that references to “one embodiment” or “an embodiment” or “an alternative embodiment” two or more times in different places in the present disclosure do not necessarily refer to the same embodiment. In addition, certain features, structures, or characteristics in one or more embodiments of the present disclosure may be properly combined.

Furthermore, it is understood by those skilled in the art that aspects of the embodiments of the present disclosure may be illustrated and described by a number of patentable varieties or circumstances, including any new and useful process, machine, product, or substance, or any combination thereof, or any new and useful improvement thereof. Accordingly, aspects of the embodiments of the present disclosure may be performed entirely by hardware, may be performed entirely by software (including firmware, resident software, microcode, etc.), or may be performed by a combination of hardware and software. The above hardware or software can be referred to as “data block”, “module”, “engine”, “unit”, “component”, or “system”. In addition, aspects of the embodiments of the present disclosure may be manifested as a computer product disposed in one or more computer-readable media comprising computer-readable program codes.

Computer storage media may contain a propagation data signal embedded with a computer program code, such as on a baseband or as part of a carrier. The propagation signal may have a variety of manifestations, including an electromagnetic form, an optical form, or the like, or suitable combinations thereof. The computer storage media may be any computer-readable media, other than the computer-readable storage media, which may be used by connecting to an instruction execution system, device, or apparatus for communicating, propagating, or transmitting for use. Program codes disposed on the computer storage media may be propagated via any suitable media, including radio, cable, fiber optic cable, RF, or the like, or any combination thereof.

Computer program codes required for the operation of the various portions of the embodiments of the present disclosure may be written in any one or more of a number of programming languages, including object-oriented programming languages such as Java, Scala, Smalltalk, Eiffel, JADE, Emerald, C++, C#, VB.NET, Python, etc., conventional procedural programming languages such as C, VisualBasic, Fortran 2003, Perl, COBOL 2002, PHP, ABAP, dynamic programming languages such as Python, Ruby and Groovy, or other programming languages, etc. The program codes can be run entirely on the user's computer, or as a stand-alone package on the user's computer, or partially on the user's computer and partially on a remote computer, or entirely on a remote computer or processing device. In the latter case, the remote computer can be connected to the user's computer through any form of network, such as a local area network (LAN) or wide area network (WAN), or connected to an external computer (e.g., via the Internet), or in a cloud computing environment, or used as a service such as software as a service (SaaS).

In addition, unless clearly stated in the claims, the sequence of processing elements and sequences described in the present disclosure, the use of counts and letters, or the use of other names are not used to limit the sequence of processes and methods in the present disclosure. While the foregoing disclosure has discussed by way of various examples some embodiments of the invention that are presently believed to be useful, it should be understood that such detail is for illustrative purposes only and that the appended claims are not limited to the disclosed embodiments, but rather, the claims are intended to cover all modifications and equivalent combinations that fall within the spirit and scope of the embodiments of the present disclosure. For example, although the implementation of various components described above may be embodied in a hardware device, it may also be implemented as a software only solution, e.g., an installation on an existing server or mobile device.

In the same way, it should be noted that in order to simplify the expression disclosed in this disclosure and help the understanding of one or more embodiments of the invention, in the foregoing description of the embodiments of the present disclosure, sometimes multiple features are combined into one embodiment, drawings or descriptions thereof. This method of disclosure does not, however, imply that the subject matter of the disclosure requires more features than are recited in the claims. Rather, claimed subject matter may lie in less than all features of a single foregoing disclosed embodiment.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described. cm What is claimed is:

1. A pre-training method for a dose distribution prediction model, implemented on a device including at least one processor and at least one storage device, the method comprising:
obtaining a plurality of training tasks;
for each of the plurality of training tasks, obtaining one or more samples corresponding to the training task; and
obtaining a pre-training model based on the samples corresponding to the plurality of training tasks, the pre-training model being configured to obtain a dose distribution prediction model for a target task by adjusting, based on one or more samples corresponding to the target task, the pre-training model, the dose distribution prediction model being configured to output predicted dose distribution information corresponding to the target task; wherein for each of the samples of the plurality of tasks and the target task, a label of the sample includes labeled dose distribution information corresponding to the task.

2. The pre-training method of claim 1, wherein the pre-training model is obtained through a meta-learning method.

3. The pre-training method of claim 1, wherein
each of at least one of the plurality of training tasks corresponds to a subject or a group of subjects; and
for each of the one or more samples corresponding to the training task, a model input of the sample includes reference information of the subject or the group of subjects, and the label of the sample includes the labeled dose distribution information of the subject or the group of subjects.

4. The pre-training method of claim 1, wherein
each of at least one of the plurality of training tasks corresponds to a lesion type; and
for each of the one or more samples corresponding to the training task, a model input of the sample includes an image including a lesion of the lesion type, and the label of the sample includes the labeled dose distribution information of the lesion.

5. The pre-training method of claim 1, wherein
each of at least one of the plurality of training tasks corresponds to a weight combination, the weight combination includes a planning target volume (PTV) weight and an organ at risk (OAR) weight, the PTV weight indicates a dose requirement for a PTV, and the OAR weight indicates a dose requirement for an OAR; and
for each of the one or more samples corresponding to the training task, a model input of the sample includes the weight combination corresponding to the sample, and the label of the sample includes the labeled dose distribution information that meets the weight combination corresponding to the sample.

6. The pre-training method of claim 1, wherein
each of at least one of the plurality of training tasks corresponds to at least one body part; and
for each of the one or more samples corresponding to the training task, a model input of the sample includes an image of the at least one body part, and the label of the sample includes the labeled dose distribution information of the at least one body part.

7. The pre-training method of claim 6, wherein when the one of the plurality of training tasks corresponds to two or more body parts, the image of the two or more body parts is obtained by a process including:
obtaining a sample image corresponding to each of the two or more body parts; and
determining the image of the two or more body parts by performing image composition on the sample images of the two or more body parts.

8. The pre-training method of claim 6, wherein when the one of the plurality of training tasks corresponds to two or more body parts, for each of the one or more samples corresponding to the training task, the model input of the sample further includes feature information corresponding to the two or more body parts.

9. The pre-training method of claim 8, wherein the feature information includes at least one of a type of each of the two or more body parts, a distance between the two or more body parts, a weight of each of the two or more body parts, a parameter of a simulated treatment device, or a subject feature corresponding to each of the two or more body parts.

10. The pre-training method of claim 1, wherein an input of the dose distribution prediction model includes at least one of:
at least one image of at least one body part of a target subject, contour information of a PTV of the target subject, contour information of an OAR of the target subject, a dose requirement for the PTV, or a dose requirement for the OAR.

11. The pre-training method of claim 1, further comprising:
obtaining a treatment prediction model for a target task by adjusting, based on the one or more samples corresponding to the target task, the pre-training model, wherein the treatment prediction model for the target task is configured to output a predicted treatment result and/or a risk of complication corresponding to the target task.

12. A pre-training system for a dose distribution prediction model, comprising:
at least one storage device including a set of instructions; and
at least one processor in communication with the at least one storage device, wherein when executing the set of instructions, the at least one processor is directed to perform operations including:
obtaining a plurality of training tasks;
for each of the plurality of training tasks, obtaining one or more samples corresponding to the training task; and
obtaining a pre-training model based on the samples corresponding to the plurality of training tasks, the pre-training model being configured to obtain a dose distribution prediction model for a target task by adjusting, based on one or more samples corresponding to the target task, the pre-training model, the dose distribution prediction model being configured to output predicted dose distribution information corresponding to the target task; wherein for each of the samples of the plurality of tasks and the target task, a label of the sample includes labeled dose distribution information corresponding to the task.

13. The pre-training system of claim 12, wherein the pre-training model is obtained through a meta-learning method.

14. The pre-training system of claim 12, wherein
each of at least one of the plurality of training tasks corresponds to a subject or a group of subjects; and
for each of the one or more samples corresponding to the training task, a model input of the sample includes reference information of the subject or the group of subjects, and the label of the sample includes the labeled dose distribution information of the subject or the group of subjects.

15. The pre-training system of claim 12, wherein
each of at least one of the plurality of training tasks corresponds to a lesion type; and
for each of the one or more samples corresponding to the training task, a model input of the sample includes an image including a lesion of the lesion type, and the label of the sample includes the labeled dose distribution information of the lesion.

16. The pre-training system of claim 12, wherein
each of at least one of the plurality of training tasks corresponds to a weight combination, the weight combination includes a planning target volume (PTV) weight and an organ at risk (OAR) weight, the PTV weight indicates a dose requirement for a PTV, and the OAR weight indicates a dose requirement for an OAR; and
for each of the one or more samples corresponding to the training task, a model input of the sample includes the weight combination corresponding to the sample, and the label of the sample includes the labeled dose distribution information that meets the weight combination corresponding to the sample.

17. The pre-training system of claim 12, wherein
each of at least one of the plurality of training tasks corresponds to at least one body part; and
for each of the one or more samples corresponding to the training task, a model input of the sample includes an image of the at least one body part, and the label of the sample includes the labeled dose distribution information of the at least one body part.

18. The pre-training system of claim 17, wherein when the one of the plurality of training tasks corresponds to two or more body parts, the image of the two or more body parts is obtained by a process including:
obtaining a sample image corresponding to each of the two or more body parts; and
determining the image of the two or more body parts by performing image composition on the sample images of the two or more body parts.

19. The pre-training system of claim 12, wherein an input of the dose distribution prediction model includes at least one of
at least one image of at least one body part of a target subject, contour information of a PTV of the target subject, contour information of an OAR of the target subject, a dose requirement for the PTV, or a dose requirement for the OAR.

20. A training method for a dose distribution prediction model, implemented on a device including at least one processor and at least one storage device, the method comprising:
obtaining a pre-training model;
obtaining one or more samples corresponding to a target task, a label of each of the one or more samples corresponding to the target task including labeled dose distribution information corresponding to the task; and
obtaining a dose distribution prediction model for the target task by adjusting, based on the one or more samples, the pre-training model, the dose distribution prediction model for the target task being configured to output predicted dose distribution information corresponding to the target task.
